(19) United States
(12) Patent Application Publication
(10) Pub. No.: US 2025/0072442 A1
(43) Pub. Date: Mar. 6, 2025

(54) FERMENTED PLANT-BASED COMPOSITIONS AND PROCESSES OF PREPARING THE SAME

(71) Applicant: Compagnie Gervais Danone, Paris (FR)

(72) Inventors: Mark SPRINGETT, Limours (FR); Laetitia FLOTTES DE POUZOLS, Rueil Malmaison (FR)

(21) Appl. No.: 18/723,845

(22) PCT Filed: Dec. 22, 2022
(86) PCT No.: PCT/EP2022/087495

§ 371 Date: Jun. 24, 2024

(30) Foreign Application Priority Data
Dec. 24, 2021 (EP) 21315294.5

(51) Int. Cl. A23C 11/10; A23J 3/16; A23J 3/34; C12N 9/48; C12N 9/52

(52) U.S. Cl. CPC A23C 11/106; A23J 3/16; A23J 3/346; C12N 9/485; C12N 9/52

(57) ABSTRACT

The present invention relates to a plant-based composition comprising a mixture comprising: i) a plant base comprising at least 5% w/w protein, at least 1% w/w fat: ii) at least one protease wherein the plant-based composition has a viscosity greater than 800 mPa·s.

### CROSS-REFERENCE TO RELATED APPLICATIONS

None

### FIELD OF THE INVENTION

The present invention relates to fermented plant-based compositions, and processes of preparing the same. More specifically, the present disclosure is directed to plant-based food products that have improved viscosity, fermentability and organoleptic qualities.

### BACKGROUND OF THE INVENTION

There is increased interest in plant-based diets among mainstream consumers who consider themselves vegan, vegetarian or flexitarian. To cater to the dietary needs of such consumers a wide variety of plant-based analogues or alternatives to non-vegan food products are increasingly available. These include plant-based dairy alternatives such as milks, yogurts, cheeses & frozen desserts. The formulation of such products to provide a textural, sensory and/or nutritional equivalent to dairy product remains challenging.

In particular, the addition of protein to provide high protein or dairy equivalent levels of protein can be challenging from a flavor perspective because vegetal proteins, such as pea or soy proteins, are often associated with flavor off-notes. Furthermore, the addition of protein is also challenging from a texture point of view, particularly when used in fermented products such as plant-based dairy alternative, such as yogurts (“PBAYs”). Indeed, fermentation leads to a reduction in pH which when it reaches the isoelectric point can result in precipitation and unwanted grainy textures, this is further exacerbated by protein-protein interactions that can result in “clumping”.

The textural challenge can be addressed using commonly used ingredients such as pectin which act as “smoothing” agents in plant-based yogurt alternatives or by including a smoothing step in the process of preparation of the food products. However, these solutions do not make it possible to obtain high protein food product and consumers increasingly prefer food products with a short and familiar ingredient list (so-called “clean label”).

Thus, there is a need in the art to find a solution to improve the texture and organoleptic properties of protein rich fermented plant-based food products with a minimum use of additives.

### SUMMARY OF THE INVENTION

The present invention arises by the unexpected finding by the inventors that the use of at least one protease in a plant-based dairy alternative comprising at least 5% of soy protein strongly reduced the viscosity due to the plant-protein and gives a dairy alternative with an improved texture in mouth, taste, creaminess and less syneresis. In addition, the step of fermentation during the preparation of the dairy alternative has been improved and the fermentation time reduced.

Thus, the present invention relates to a fermented plant-based composition comprising:
i) a plant base comprising at least 5% w/w protein, at least 1% w/w fat;
ii) at least one protease;
wherein the plant-based composition has a viscosity greater than 800 mPa·s.

The present invention also relates to a process for the preparation of a fermented plant-based composition comprising the steps of:
a) proving a mixture comprising a plant base comprising at least 5% w/w protein and at least 1% w/w fat;
b) adding at least one protease;
c) fermenting the mixture to provide a fermented plant-based composition having a viscosity greater than 800 mPa·s.

The present invention also relates to a fermented plant-based composition obtained by the process as defined above.

The present invention also relates to a plant-based alternative dairy alternative comprising a fermented plant-based composition as defined above.

### DETAILED DESCRIPTION OF THE INVENTION

### Definitions

In the specification and in the claims, the terms “including”, “comprising” and “containing” can be used interchangeably. These terms are open-ended terms and should be interpreted to mean “including”, but not limited to. Thus, when an object “comprises” or “contains” one or several elements, other elements than those mentioned may also be included in the object. These terms encompass the more restrictive terms “consisting essentially of” and “consisting of.” When an object is said to “consist of” one or several elements, the object is limited to the listed elements and cannot include other elements than those mentioned.

It must be noted that as used herein and in the appended claims, the singular forms “a”, “an”, and “the” include plural references unless the context clearly dictates otherwise. As well, the terms “a”, “an”, “one or more” and “at least one” can be used interchangeably herein.

As used herein, the term “ppm” shall be taken to mean “parts per million”. One milliliter in 1 liter is 1000 ppm and one thousandth of a milliliter (0.001 g) in 1 liter is one ppm.

As used herein, the terms “x % (w/w)” and “x % w/w” are equivalent to “x g per 100 g”. Unless indicated otherwise, all % value shall be taken to indicate x % w/w.

As used herein, the term “x % (v/v)” “x % v/v” is equivalent to “x ml per 100 ml”.

In the context of this application, the term “at least” also includes the starting point of the open range. For example, an amount of “at least 95.00% w/w” means any amount equal to 95.00 percentage by weight or above.

In the context of this application, the term “about” defines a range of plus or minus 10% of the cited value. For example, an amount of “about 20 weight %” means any amount within the range of 18.00 to 22.00 weight %.

As used herein, the terms “vegan” or “plant-based” shall be taken to mean a composition or product which comprises plant or plant-derived matter but which does not comprise animal or animal-derived matter, including but not limited to dairy, egg, fish, shellfish, meat, dairy milk and insects.

As used herein the term “vegetal” shall be taken to mean edible parts of a plant including but not limited to vegetables, fruits, flowers, stems, seeds, leaves and roots.

As used herein, the term “dairy” shall be taken to mean a composition or product comprising or consists of mammalian milk matter, i.e., the lacteal secretion obtainable by milking.

As used herein, the terms “free” or “free from” shall be taken to mean that a given substance is not added to make a composition or product but, unless specified, where trace amounts or contaminants thereof may be present.

As used herein, the term “added sugar” shall refer to sugars that are added during the production or processing of foods (e.g., refined sugars that may be added to a vegetal base of processed plant matter) as opposed to sugars naturally occurring in said foods. Added sugars include sugars (free, mono- and disaccharides), sugars from syrups (e.g., rice or cereal syrups) and honey, and sugars from concentrated fruit or vegetable juices that are in excess of what would be expected from the same volume of 100 percent fruit or vegetable juice of the same type.

As used herein, the terms plant-based alternative, analogue or substitute shall be taken to mean a plant-based food or beverage composition that is formulated to simulate the organoleptic and/or functional qualities of a non-plant-based product.

The “dry matter” of a product corresponds to the weight of non-volatile components present in the product relatively to the total weight of the product. The dry matter is expressed as a weight percentage. The “non-volatile components” correspond to the solids that remain after total evaporation of water of the product.

As used herein, the term “added protein” shall refer to protein ingredients that are added during the production or processing of foods (e.g., protein isolates or concentrates that may be added to a plant base of processed plant matter) as opposed to sugars naturally occurring in said matter. Added proteins include protein concentrates, protein isolates and protein hydrolysate ingredients that are in excess of what would be expected from the same volume of 100 percent plant base of the same type.

As used herein, the term “fermented plant-based” shall be taken to mean a product or composition that is the product of the acidifying fermentation of a plant-based composition by a starter culture of fermenting microorganisms, in particular bacteria, preferably lactic acid bacteria.

As used herein the term “strained fermented food product” or “concentrated fermented food product” shall be taken to mean a food product which has been subjected to a concentration process before, during or after fermentation.

As used herein the term “spoonable” shall be taken to mean a solid or semi-solid that may be consumed by means of a spoon or other utensil.

As used herein, the “fat content” of a composition corresponds to the weight of the fat components present in the composition relatively to the total weight of the composition. The fat content is expressed as a weight percentage.

The expression “protein concentrate” as used herein, generally refers to protein derived from a plant source that has been extracted from the plant source and purified. Protein concentrate may comprise up to about 60%, 70% or more total protein on a dry matter basis e.g., 50% up to 70% w/w. Preferably, the rest of the dry matter of the vegetal protein concentrate comprises carbohydrates such as starch and fibers. A protein concentrate may comprise a single type of protein or a combination of different types of proteins. A protein concentrate may comprise a protein from a single vegetal variety or a combination of different vegetal varieties. A protein concentrate can be deflavored or not.

The expression “protein isolate” as used herein, generally refers to protein derived from plant source that has been extracted from the plant source and purified. Protein isolate may comprise greater than or equal to about 70%, 75%, 80%, 85% 90% w/w, or more total protein on a dry matter basis e.g., 70% to 95% w/w. A protein isolate may comprise a single type of protein or a combination of different types of proteins. A protein isolate may comprise a protein from a single vegetal variety or a combination of different vegetal varieties. A protein isolate may comprise at least one protein isolate which has been hydrolyzed and/or deflavored. By way of example a protein isolate may comprise a combination of at least one protein isolate and at least one protein isolate which has been hydrolyzed.

As intended herein, the expression “hydrolyzed proteins” refers to proteins which have been hydrolyzed into their amino acids and peptides components. The hydrolysis of the proteins may be carried out by any method well known to the person skilled in the art. By way of example, the hydrolysis can be accomplished by a chemical process, e.g., by adding the protein in an acid-based solution or using various enzymes such as proteases.

As intended herein, the expression “deflavoured” refers to proteins which have been treated to reduce or remove off-notes such as bitterness or to provide a neutral tasting food preparation ingredient.

### Plant-Based Composition

Preferably, the plant-based composition, in particular the fermented plant-based composition, according to the present embodiments is particularly suited for use in a food product, in particular a plant-based dairy alternative such as yogurt. Thus, embodiments provide a fermented plant-based composition suitable for consumption.

Embodiments provide a fermented plant-based food product, in particular a plant-based dairy alternative, comprising a fermented plant-based composition according to the present embodiments.

Embodiments also provide a fermented plant-based composition as defined above for use as or in a plant-based dairy alternative or in a plant-based alternative yogurt.

### Plant-Based Food Product

The plant-based composition, in particular the fermented plant-based composition, according to the present embodiments is preferably a fermented plant-based dairy alternative, in embodiments selected from the group consisting of a spoonable composition, a viscous drink, a plant-based alternative to yogurt, a plant-based alternative to set yogurt, a plant-based alternative to stirred yogurt, a plant-based alternative to strained yoghurt, a plant-based skyr, a plant-based alternative to pourable yogurt, a plant-based alternative to drink yogurt, a plant-based alternative to frozen yogurt, a kefir, a buttermilk, a plant-based alternative to quark, a plant-based alternative to sour cream, a plant-based alternative to fresh cheese, a plant-based alternative to cheese, a smoothies etc.

Preferably, the plant-based composition, in particular the fermented plant-based composition, according to the present embodiments is selected from the group consisting of plant-based dairy alternative based on or comprising soy, almond, rice, chia, oat, pea, hazelnut, nut, wheat, quinoa, buckwheat, cashew, pecan, macadamia, pistachio, walnuts, hemp, pumpkin, sesame, tiger nut, sunflower, coconut, lupin, faba and/or combinations thereof.

### Viscosity

Preferably, the plant-based composition, in particular the fermented plant-based composition, according to the present embodiments has a viscosity greater than 800 mPa·s, more preferably greater than 900 mPa·s, more preferably greater than 1000 mPa·s, more preferably greater than 1500 mPa·s, more preferably greater than 2000 mPa·s, more preferably greater than 2500 mPa·s, more preferably greater than 3000 mPa·s, more preferably greater than 3500 mPa·s, more preferably greater than 4000 mPa·s, more preferably greater than 4500 mPa·s at 10° C., at a shear rate of 64 s⁻¹. In embodiments, the plant-based composition has a viscosity range of 800 to 1000 mPa·s, 900 to 1000 mPa·s, 900 to 1200 mPa·s, 900 to 1500 mPa·s, 900 to 2000 mPa·s at a shear rate of 64 s⁻¹. In embodiments, the plant-based composition has a viscosity range of 1000 to 2000 mPa·s, 1000 to 2200 mPa·s, 1000 to 2500 mPa·s, 1500 to 2500 mPa·s, 1500 to 3000 mPa·s, 1500 to 4000 mPa·s, 1500 to 4500 mPa·s at a shear rate of 64 s⁻¹. The viscosity can be measured by any method well known by the person skilled in the art. By way of example, it is possible to use a Rheometer at a shear rate of 64 s⁻¹ during 10 to 90 s at 10° C.

### Protein and Fat

Preferably, the plant-based composition, in particular the fermented plant-based composition, according to the present embodiments comprises at least 3% w/w protein, more preferably at least 5% w/w protein. Preferably, the plant-based composition, in particular the fermented plant-based composition, according to the present embodiments comprises up to 20% w/w protein. By way of example, the plant-based composition according to the present embodiments comprises 3 to 20% w/w, from 5 to 20% w/w, or from 5 to 18% w/w, or from 5 to 15% w/w, or from 5 to 12% w/w, or from 5 to 11.5% w/w, or from 5 to 11% w/w, or from 5 to 10% w/w, or from 5 to 9% w/w, or from 5 to 8% w/w. More preferably the plant-based composition, in particular the fermented plant-based composition, according to the present embodiments comprises from 3% to 12% or from 5% to 12% w/w protein, even more preferably from 7 to 10% w/w protein, or from 8 to 10% w/w and most preferably about 10% w/w protein.

Preferably, the plant-based composition, in particular the fermented plant-based composition, according to the present embodiments comprises at least 0.75%, or at least 1% fat. Preferably, the plant-based composition, in particular the fermented-plant-based composition, according to the present embodiments comprises up to 12% w/w fat. By way of example, the plant-based composition, in particular the fermented plant-based composition, according to the present embodiments comprises from 1 to 3% w/w, or from 3 to 4% w/w, or from 3 to 5% w/w, or from 3 to 5.5% w/w, or from 3 to 6% w/w, or from 3 to 10% w/w fat.

### Plant Base

Preferably, the plant base comprising protein and fat, according to the present embodiments comprises at least one type of plant-matter and can be in any form such as a liquid, a suspension, in particular an aqueous suspension or a slurry.

Preferably, the plant-matter is selected from the group consisting of legumes, pulses, nuts, seeds, cereals and/or combinations thereof. Preferably, the plant-matter according to the invention comprises protein and fat. By way of example, the plant-matter may be in the form of a vegetable milk, a vegetable butter, a powder, a vegetable juice, a retentate, a flour, milled, ground, soaked, dehulled, a lyophilized powder, a freeze-dried powder, a powder, a mixture of powder and water, a mixture of powder and milk, optionally enzymatic hydrolyzed and/or homogenized and/combinations thereof etc.

The pulses according to the present embodiments can be chosen among any pulses well known by the person skilled in the art. Preferably, the pulses according to the present embodiments are selected from the group consisting of split peas, field peas, dry peas, lentil, chickpeas, garbanzo bean, faba beans, konda, navy bean, white navy bean, white pea bean, pea bean, cow pea, horse bean, haricot, pinot bean, mottled bean, small red bean, red Mexican bean, kidney bean, black bean, black turtle bean, cranberry bean, roman bean, speckled sugar bean, lima bean, faba bean, Madagascar bean, green gram, mung bean, green bean, black gram, urad dal, soy, lupin and combinations thereof. In preferred embodiments, the pulses according to the present embodiments are selected from the group consisting of pea, bean faba bean, soy, chickpea and combinations thereof.

The nuts according to the present embodiments can be chosen among any nuts well known by the person skilled in the art. Preferably, the nuts are selected from the group consisting of almonds, cashews, pecans, macadamias, hazelnuts, pistachio, walnuts and combinations thereof. In preferred embodiments, the nut is almond.

The seeds according to the present embodiments can be chosen among any seeds well known by the person skilled in the art. Preferably, the seeds according to the present embodiments are selected from the group consisting of hemp, pumpkin, quinoa, sesame, tiger nut, flax, chia, sunflower, coconut and combinations thereof.

The cereals according to the present embodiments can be chosen among ay cereals well known by the person skilled in the art. Preferably, the cereals according to the present embodiments are selected from the group consisting of wheat, rye, spelt, barley, oat, millet, sorghum, rice, teff and combinations thereof. In preferred embodiments the cereal is oat.

By way of example, the plant base may be an aqueous suspension comprising water and plant matter. Processes for the preparation of such aqueous suspensions are known in the art and typically comprise mechanical and/or enzymatic disruption of the plant-matter and hydration and/or combination with a solution, followed by mechanical separation of an aqueous fraction from starchy and/or fibrous matter, e.g., by decanting, centrifugation or filtration.

By way of example, the plant base may comprise a pulse, a seed or a nut butter such as sunflower, sesame, soy, almond, cashew, hazelnut or peanut butter. Processes for the preparation of butters typically comprise wet or dry grinding roasted or unroasted pulses, seed or nuts to a paste having a particle size suitable for the preparation of beverages.

By way of example, the plant base may comprise a hydrolyzed cereal suspension such as an oat milk or syrup. Processes for the preparation of such cereal suspensions typically comprise mixing an oat material (such as rolled oats, milled oats, oat flour or oatmeal) with water and treated enzymatically by amylases to hydrolyze starch followed by removal of suspended matter.

In an embodiment, the plant base comprises a powder, in particular a powder of legumes, pulses, nuts, seeds or cereals and a vegetable milk of legumes, pulses, nuts, seeds or cereals, such as for example a mixture of soy powder and soy milk. As used herein, the expression soy milk is equivalent to soy juice.

In an embodiment, the plant base comprises a powder, in particular a powder of legumes, pulses, nuts, seeds or cereals and water.

In an embodiment, the plant base is a vegetable milk, such as for example soy milk, almond milk, rice milk, chia milk, oat milk, pea milk, hazelnut milk, nut milk, wheat milk, quinoa milk, buckwheat milk, cashew milk, pecan milk, macadamia milk, pistachio milk, walnuts milk, hemp milk, pumpkin milk, sesame milk, tiger nut milk, coconut milk, lupin milk, faba milk, etc.

In an embodiment, the plant base comprises a mixture of at least one vegetable powder, in particular a vegetable milk powder and at least one vegetable milk of legumes, pulses, nuts, seeds or cereals, such as for example a mixture of soymilk powder and soy milk.

In an embodiment, the plant base comprises a retentate of a juice of legumes, pulses, nuts, seeds or cereals, such as for example a retentate of soy juice. Method for obtaining a retentate are well known by the person skilled in the art. By way of example, the retentate may be obtained by a process of filtration of a juice of legumes, pulses, nuts, seeds or cereals. As intended herein, the retentate is the phase which does not pass through the filter.

In an embodiment, the plant base comprises a mixture of powder, in particular a vegetable milk powder of legumes, pulses, nuts, seeds or cereals and a retentate of a juice of legumes, pulses, nuts, seeds or cereals, such as for example a mixture of soymilk powder and soy juice retentate.

By way of example, the plant base comprises a mixture of a vegetable milk of legumes, pulses, nuts, seeds or cereals and a retentate of a juice of legumes, pulses, nuts, seeds or cereals, such as for example a mixture of soy milk and soy juice retentate.

As intended herein, the protein according to the present embodiments are preferably vegetal protein, i.e., protein derived from a plant source. The protein according to the present embodiments, preferably come from the plant-matter of the plant base as defined above. In particular, the protein according to the present embodiments is preferably selected from the group consisting of soy protein, pulse protein, cereal protein, nut protein and combinations thereof. More preferably, the protein according to the present embodiments is selected from the group consisting of soy protein, pea protein, faba protein, almond protein, oat protein and combinations thereof. The protein according to the present invention, may comprise at least one protein isolate, at least one protein concentrate, and combinations thereof. The protein according to the present invention, may also comprise a mixture of protein derived from the same or different plant sources.

The expression “protein concentrate” as used herein, generally refers to protein derived from a plant source that has been extracted from the plant source and purified. Protein concentrate may comprise up to about 60%, 70% or more total protein on a dry matter basis preferably, protein concentrate comprises from 50% to 70% w/w or from 50% to 69% total protein on a dry matter basis. Preferably, the rest of the dry matter of the vegetal protein concentrate comprises carbohydrates such as starch and fibers. A protein concentrate may comprise a single type of protein or a combination of different types of proteins. A protein concentrate may comprise a protein from a single vegetal variety or a combination of different vegetal varieties. A protein concentrate can be deflavored or not.

The expression “protein isolate” as used herein, generally refers to protein derived from plant source that has been extracted from the plant source and purified. Protein isolate may comprise greater than or equal to about 70%, 75%, 80%, 85% 90% w/w, or more total protein on a dry matter basis e.g., 70% to 95% w/w. A protein isolate may comprise a single type of protein or a combination of different types of proteins. A protein isolate may comprise a protein from a single vegetal variety or a combination of different vegetal varieties. A protein isolate may comprise at least one protein isolate which has been hydrolyzed and/or deflavored. By way of example a protein isolate may comprise a combination of at least one protein isolate and at least one protein isolate which has been hydrolyzed.

In embodiments of the present invention, the protein of the plant base according to the invention is not hydrolyzed or partially hydrolyzed before the addition of at least one endoprotease and at least one exoprotease. Preferably, at least a part of the protein and fat of the plant base according to the present embodiments is not in the form of added protein. Preferably, at least 50% w/w, at least 55% w/w, at least 60% w/w, at least 65% w/w, at least 70% w/w, at least 75% w/w of the protein of the plant base are not in the form of added protein.

In embodiments of the present invention, the protein of the plant base according to the invention is not a protein isolate or a protein concentrate or a mixture of protein isolate and protein isolate.

The protein according to the present invention, may be partially hydrolysed by the enzymatic activity of the protease of the invention.

As intended herein, the fat according to the present invention is preferably a vegetable fat, i.e., fat derived from a plant source. In an embodiment, the fat of the plant base come from the plant-matter as defined above.

As intended herein, the fat according to the present invention is preferably a vegetable fat, i.e., fat derived from a plant source. In an embodiment, the fat of the plant base come from the plant-matter as defined above.

Preferably, the fat according to the invention comes from the same plant source as the protein according to the invention.

Preferably, at least a part of the protein and fat of the plant base according to the present embodiments are aggregated together, notably at least a part of the protein are aggregated on the surface of at least one part of the fat particles or fat droplets of the plant base. As used herein, the term aggregated is equivalent to agglomerated, associated and complexed. Preferably, at least 50% w/w, at least 55% w/w, at least 60% w/w, at least 65% w/w, at least 70% w/w, at least 75% w/w of the protein of the plant base are aggregated with at least 50% w/w, at least 55% w/w, at least 60% w/w, at least 65% w/w, at least 70% w/w, at least 75% w/w of the fat of the plant base according to the invention.

Preferably, the plant base according to the invention comprises at least 5% w/w of plant protein and at least 1% w/w of plant fat. Preferably, the plant-based according to the present embodiments comprises up to 20% w/w protein and up to 12% w/w fat. By way of example, the plant base according to the present embodiments comprises from 5 to 20% w/w, or from 5 to 18% w/w, or from 5 to 15% w/w, or from 5 to 12% w/w, or from 5 to 11.5% w/w, or from 5 to 11% w/w, or from 5 to 10% w/w, or from 5 to 9% w/w, or from 5% to 8% w/w, or from 7 to 10% w/w protein. Preferably, the plant base according to the present embodiments comprises from 1 to 3% w/w, or from 3 to 4% w/w, or from 3 to 5% w/w, or from 3 to 5.5% w/w, or from 3 to 6% w/w, or from 3 to 10% w/w fat. Preferably also, the plant base according to the present embodiments comprises about 10% w/w protein and about 5% w/w fat, or about 7% w/w protein and about 4% w/w fat, or about 5% w/w protein and about 3% w/w fat or about 3% w/w protein and about 1% w/w fat.

In embodiments, the plant base further comprises at least one fat source. The fat source may be in the form of an oil, a cream, a butter, a paste, a milk, a powder, a lyophilized powder and any other form well known by the person skilled in the art.

Preferably, the fat source according to the present embodiments is selected from the group consisting of coconut oil, corn oil, almond oil, chia oil, safflower oil, soybean oils, linseed oil, soy oil, grape seed oil, hazelnut oil, rice bran oil, sunflower oil, sesame oil, açaí oil, palm oil, avocado oil, brazil nut, passion fruit oil, cashew oil, olive oil, coconut butter, almond butter, peanut butter, hazelnut butter, cashew butter, nut paste, almond paste, and/or combinations thereof.

In embodiments, the plant base may further comprise carbohydrates. Preferably, carbohydrates according to the invention are selected from sugars and fibers.

Example of sugars according to the present embodiments, include fructose, galactose, glucose, saccharose, sucrose, dextrose, maltose, fructose syrup, sugarcane syrup, and high fructose corn syrup.

By way of examples of fibers according to the present embodiments it is possible to cite fibers found in cereals, fruits, pulses, and vegetables, in particular in fruits, dark green vegetables, orange vegetables, cook dry bean, starchy vegetables, whole grains, more particularly in peas, soybeans, lupins, oats, rye, chia, barley, figs, plums, prunes, berries, bananas, apples, pears, broccoli, carrots, root tuber, root vegetable, sweet potato, nuts, almond, wheat, corn, chicory root and/or combinations thereof.

Particularly preferred is a plant base that comprises added sugar, where the total carbohydrate content of the plant base is derived from plant-matter selected from the group consisting of legumes, nuts, seeds, cereals and/or combination thereof and added sugar.

In embodiments the added sugar is selected from the group consisting of beet sugar, brown sugar, brown rice sugar, cane juice, cane syrup, cane sugar, caramel, coconut sugar, corn syrup solids, corn syrup, refined sugar, confectioner's sugar, date sugar, maple syrup, malt, molasses, raw sugar, sugar, honey, agave glucose, dextrose, saccharose etc. and/or combinations thereof.

In an embodiment, the sugar servers as nutriment for the fermentation.

### Water

The plant-based composition, in particular the fermented plant-based composition, according to the present embodiments may also comprises water.

Water is typically present in an amount balancing the amounts of other ingredients to 100% by weight. In an embodiment water is present in an amount between 10% and 80% by weight, for example, or from 60% to 90%, or from 40% to 45% or from 45% to 50% or from 50% to 55% or from 55% to 60% or from 60% to 65% or from 65% to 70% or from 70% to 75%, or from 75% to 80% or from 80% to 85% or from 80% to 85%. In one embodiment the water quality is monitored to ensure sufficiently low level of cations to ensure emulsification stability is not impacted. The water can be selected from any type of water well-known by the person skilled in the art such as tap water, demineralized water, desalinated water, water filtered by reverse osmosis, deionized water, or low ions content water. The total cation content can be from about 60 ppm (40 ppm for divalent ions and 20 ppm for monovalent ions) and the hardness of water can be 6 gram/gallon or less.

### Buffering Agent

The plant-based composition, in particular the fermented plant-based composition may further comprise a buffering or pH adjusting agent.

In an embodiment, the plant-based composition also comprises at least one buffering agent. The buffering or pH adjusting agent according to the present embodiments may be any buffering or pH adjusting agent well known to the person skilled in the art. By way of example of such agents it is possible to cite monophosphates, diphosphates, sodium mono- and bicarbonates, potassium mono -and bicarbonates, for example, potassium phosphate, dipotassium phosphate, potassium polyphosphates, sodium bicarbonate, trisodium citrate (also referred to as sodium citrate), sodium phosphate, disodium phosphate, trisodium phosphate and sodium polyphosphates, sodium bicarbonate, calcium carbonate, acidifying agent such as citric acid, and/or mixtures or combinations thereof.

Preferably, the pH of the plant base, i.e. the mixture before fermentation is comprised between 6 and 8, preferably from 6.3 to 7.5, even more preferably between 6.5 to 7.2.

Preferably, the fermented plant-based, the concentrated fermented plant-based composition, composition and the plant-based food product, according to the present embodiments have a pH comprised between 4 and 5.

Preferably, when a buffering agent is used, it is present in an amount of from 0.0001% to 5% by weight, for example, from 0.2% to 2, from 0.5 to 1.8% by weight compared to the total weight of the ingredient of the composition.

### Protease

As intended herein, the term protease is equivalent to peptidase or proteinase and relates to enzymes which are able to catalyze the breakdown of proteins into smaller polypeptides or single amino acids. Typically, proteases act by cleaving the peptide bonds within proteins by hydrolysis, a reaction where water breaks the peptide bonds between amino acids. The protease according to the present embodiments encompasses exoprotease and endoprotease.

Typically, exoproteases hydrolyze the peptide bonds between amino acids at the ends of the protein molecule and endoproteases hydrolyze the peptide bonds between amino acids in the interior of the protein molecule.

In an embodiment, the protease according to the present invention is a composition comprising at least one exoprotease and at least one endoprotease.

In an embodiment, the protease according to the present invention has both exoprotease and endoprotease activity.

In another embodiment, the protease according to the invention is a mixture of protease having exoprotease and endoprotease activity.

In embodiments the proteases of the invention may be fully or partially inactivated or denatured subsequent to hydrolysis of the proteins in the plant base

Preferably, the protease according to the present embodiments is selected from the group consisting of serine protease, cysteine protease, threonine protease, aspartic protease, glutamic protease, metalloprotease, asparagine peptide lyase, aminopeptidase, in particular a leucine aminopeptidase, and carboxypeptidase and/or combinations thereof.

The protease according to the present embodiments is preferably derived from a bacterial or a fungal strain, a plant or an animal.

Preferably, the bacterial protease according to the present embodiments is derived from a strain of Bacillus, preferably a strain of Bacillus subtilis or a strain of Bacillus licheniformis.

Preferably, the fungal protease according to the present embodiments is derived from a strain of Aspergillus, preferably a strain of aculeatus, a strain of Aspergillus niger, a strain of Aspergillus oryzae. By way of example of fungal protease according to the present embodiment it is possible to cite a leucine aminopeptidase derived from Aspergillus oryzae.

Preferably, the plant-derived protease according to the present embodiments is selected from the group consisting of papain, bromelain, actinidine, ficin and mixtures or combinations thereof. By way of example of plant-derived protease it is possible to cite a bromelain protease derived from Ananas cosmosus.

Preferably, the animal-derived protease according to the present embodiments is selected from the group consisting of protease having pepsin, trypsin, aminopeptidase, carboxypeptidase, chymotrypsin activity, and mixtures of combinations thereof.

In embodiments, the exoprotease is selected from the group consisting of carboxypeptidase leucyl peptidase, aminopeptidase and mixtures of combinations thereof.

In embodiments, endoprotease is selected from the group consisting of aspartic endopeptidases, cysteine endopeptidases, glutamic endopeptidases, metalloendopeptidases, serine endopeptidases, threonine endopeptidases, pepsin, chymotrypsine, thermolysine, trypsine and mixture or combination thereof.

### Lactic Acid Bacteria

The plant-based composition, in particular the fermented plant-based composition, according to the present embodiments further comprises at least one strain of lactic acid bacteria. The selection of suitable lactic acid bacteria strains is within the scope of the skilled person and is typically a thermophilic and/or a mesophilic lactic acid bacteria. Lactic acid bacteria may be referred to herein as ferments or cultures or starters. Examples of lactic acid bacteria that can be used include but are not limited to Lactobacilli (for example Lactobacillus acidophilus, Lactobacillus buchneri, Lactobacillus delbrueckii, in particular L. delbrueckii subsp. bulgaricus or lactis, Lactobacillus casei, Lactobacillus plantarum, Lactobacillus reuteri, Lactobacillus johnsonii, Lactobacillus helveticus, Lactobacillus brevis, Lactobacillus rhamnosus); Lactococci (for example Lactococcus lactis, typically Lactococcus lactis subsp. lactis or Lactococcus lactis subsp. cremoris), Streptococci (for example, Streptococcus thermophilus, Streptococcus cremoris) and mixtures and/or combinations thereof. Typically, a mixture or association of a plurality of species of lactic acid bacteria may be used, typically a mixture or association of Lactobacillus and Streptococcus. For the preparation of plant-based food product thereto this typically includes Lactobacillus bulgaricus (also referred to as Lactobacillus delbrueckii subsp. bulgaricus). The lactic acid bacteria may be introduced in any appropriate form, for example, in a spray-dried form, a freeze-dried form or in a frozen form, preferably in a liquid form. The optimization of the amount of lactic acid bacteria is well within the scope of the person skilled in the art. Preferably, the lactic acid bacteria may decrease the pH of the plant-based composition during fermentation to 4.2-4.3.

### Intermediate Preparation

The plant-based composition, in particular the fermented plant-based composition, according to the present embodiments can further comprises an intermediate preparation. Intermediate preparations are well known by the person skilled in the art. They are typically used to modify the taste, mouthfeel and/or texture of a food product, for example of a fermented food product. They can also be used to introduce some additives such as nutrients in the plant-based composition.

Intermediate preparation can be in any form suitable with a plant-based composition, in particular a fermented plant-based composition, more particularly a fermented plant-based food product. By way of example, the intermediate preparation according to the present embodiments can be a slurry or a fruit preparation.

Preferably, the intermediate preparation according to the present embodiments comprises at least one selected from the group consisting of water, flavoring agent, sweetener, fruit or fruit preparation, stabilizing agent, coloring agents, vitamin, oilseeds, nuts, cereals, and/or combination thereof.

Advantageously, the flavoring agent is used to enhance and/or change the taste of the composition. The flavoring agent according to the present embodiments can be any flavoring agent well known to the person skilled in the art e.g., fruit flavors such as citrus flavor, strawberry flavor, peach flavor, apricot flavor, raspberry flavor, orange flavor, apple flavor, pear flavor, etc., vanilla flavors, caramel flavors, coffee flavors, almond flavors, and chocolate flavors.

Advantageously, sweeteners are added to enhance the taste of the flavorings provided and/or provide overall sweetness to the plant-based composition. Sweeteners according to the present embodiments can be chosen among any sweetener well known to the person skilled in the art such as for example sugar, fructose, glucose, maltose, sucrose, dextrose, high fructose corn syrup, sugarcane syrup, sucralose, acesulfamK, aspartam, saccharine, sugar substitute like sorbitol, mannitol, xylitol, combination thereof.

Preferably, the sweetener according to the present embodiments is present in the plant-based composition in an amount between 1 and 10% w/w relative to the total weight of the ingredient of the composition.

The ingredients and/or components of the intermediate preparation and the amounts thereof can be typically such that the composition has a brix degree of from 1 to 65 brix, for example from 1 to 10 brix, or from 10 to 15 brix, or from 15 to 20 brix, or from 20 to 25 brix, or from 25 to 30 brix, or from 30 to 35 brix, or from 35 to 40 brix, or from 40 to 45 brix, or from 45 to 50 brix, or from 50 to 55 brix, or from 55 to 60 brix, or from 55 to 60 brix, or from 60 to 65 brix. The Brix degree corresponds to the sugar content of a composition. Methods for measuring the brix degree are well-known by the person skilled in the art. For example, when measuring the brix degree of a fruit preparation, the fruit preparation is filtered on a sieve of 1 mm, and the supernatant (thus, without fruit pieces) is collected.

The stabilizing agent according to the present embodiments can be any stabilizing agent well known by the person skilled in the art. They typically help in avoiding phase separation of solids, for examples of fruits or fruits extracts and/or in avoiding syneresis. The stabilizing agent can for example be a starch, a pectin, a guar, a xanthan, a carrageenan, a locust bean gum, a gellan gum or a mixture thereof. The amount of stabilizing system is typically of from 0.5 to 5% by weight.

In a particularly preferred embodiment the plant-based composition, in particular the fermented plant-based composition, according to the present embodiments are substantially free of stabilizing agents. Should the composition comprise a minor amount of a stabilizing agent, this amount is less than 0.1% by weight, for example, less than 0.05%, for example, less than 0.01%, for example, less than 0.005%, for example, less than 0.001%.

The intermediate preparation according to the present embodiments may further comprise at least one vitamin preferably selected from vitamin A, D, B2, B12 and combination thereof.

In an embodiment, the intermediate preparation comprises a fruit or a fruit preparation. As used herein the term “fruit” refers to any fruit form, including for example full fruits, pieces, purees, concentrates, juices etc. Examples of fruits include for example strawberry, peach, apricot, mango, apple, pear, raspberry, blueberry, blackberry, passion, cherry, pineapple, banana, fig, prune, coconut, quince, kiwi, and mixtures or associations thereof, such as peach-passion.

The fruits can be for example provided as:
frozen fruit cubes, for example 10 mm fruit cubes, for example Individual Quick Frozen fruit cubes, for example strawberry, peach, apricot, mango, apple, pear fruit cubes or mixtures thereof;
Aseptic fruit cubes, for example 10 mm fruit cubes, for example strawberry, peach, apricot, mango, apple or pear fruit cubes or mixtures thereof;
fruit purees, for example fruit purees concentrated from 2 to 5 times, preferably 3 times, for example aseptic fruit purees, for example strawberry, peach, apricot, mango, raspberry, blueberry or apple fruit purees or mixtures thereof;
single aseptic fruit purees, for example strawberry, raspberry, peach, apricot, blueberry or apple single aseptic fruit purees or mixture thereof;
frozen whole fruits, for example Individual Quick Frozen whole fruits, for example blueberry, raspberry or blackberry frozen whole fruits, or mixtures thereof;
   and/or mixtures thereof.

A fruit preparation according to the present embodiments can for example comprise fruit in an amount of from 30 to 80% by weight for example from 50 to 70% by weight.

The fruit preparation can comprise pH modification agents such as citric acid. The fruit preparation can have a pH of from 2.5 to 5, preferably of from 2.8 to 4.2.

Oilseed, nut and cereal are for example slurries. Oilseed slurries according to the present embodiments can comprise almond paste, pistachio paste, hazelnuts past, nut paste, etc. Cereal slurries according to the present embodiments can comprise oat flour, oat syrup, etc.

Typically, a fruit preparation or slurries, in particular oilseed or cereal slurries, can be added in an amount of 1-30% by weight with reference to the total amount of the plant-based composition or for example from 1% to 25% w/w, from 1% to 20% w/w, from 1% to 15% w/w, from 1% to 10% w/w.

In embodiments the plant-based composition, in particular the fermented-plant-based composition, according to the present embodiments comprises up to about 30% w/w of said intermediate preparation, e.g., up to about 10%, 15%, 20%, 25% (w/w). In one embodiment, the plant-based composition, or the plant-based food product according to embodiments of the present invention comprise 0% to 30% w/w of said intermediate preparation. In alternative embodiments, the plant-based composition, in particular the fermented plant-based composition, comprise 1% to 25% w/w of said intermediate preparation. In further alternative embodiments, the plant-based composition, in particular the fermented plant-based composition, according to embodiments of the invention comprise 1% to 20% w/w of said intermediate preparation. In additional embodiments, the plant-based composition or plant-based food product according to embodiments of the invention comprise 1% to 15% w/w of said intermediate preparation. In further additional embodiments, the plant-based composition or plant-based food product according to embodiments of the invention comprise 1% to 10% w/w of said intermediate preparation.

### Packaging

Preferably, the plant-based composition, in particular the fermented plant-based composition, more particularly the plant-based food product, according to embodiments of the invention is provided in a sealed or sealable container, or in a bottle, or in a can containing about 50 g, 60 g, 70 g, 75 g, 80 g, 85 g, 90 g, 95 g, 100 g, 105 g, 110 g, 115 g, 120 g, 125 g, 130 g, 135 g, 140 g, 145 g, 150 g, 200 g, 300 g, 320 g or 500 g or 750 g, or 900 g, or 1000 g about 1 oz, 2 oz, 3 oz, 4 oz, 5 oz, 6 oz, 12 oz, 15 oz, 17 oz, 20 oz, or 35 oz or about 150 ml, 200 ml, 220 ml, 230 ml, 240 ml, 250 ml, 300 ml, 320 ml, 330 ml, 340 ml, 350 ml, 370 ml, 400 ml, 440 ml, 470 ml, 500 ml, 550 ml, 1000 ml, or 1500 ml of product.

In embodiments, the plant-based composition, in particular the fermented plant-based composition, more particularly the plant-based food product, according to embodiments of the invention is provided in a sealed or sealable container, or a bottle, or a can containing about 50 g to 500 g, 60 g to 500 g, 70 g to 500 g, 75 g to 500 g, 80 g to 500 g, 85 g to 500 g, 90 g to 500 g, 95 g to 500 g, 100 g to 500 g, 105 g to 500 g, 110 g to 500 g, 115 g to 500 g, 120 g to 500 g, 125 g to 500 g, 130 g to 500 g, 135 g to 500 g, 140 g to 500 g, 145 g to 500 g, 150 g to 500 g, 200 g to 500 g, 300 g to 500 g, 320 g to 500 g or 500 g or about 150 ml to 1000 ml, 200 ml to 1000 ml, 220 ml to 1000 ml, 230 ml to 1000 ml, 240 ml to 1000 ml, 250 ml to 1000 ml, 300 ml to 1000 ml, 320 ml to 1000 ml, 330 ml to 1000 ml, 340 ml to 1000 ml, 350 ml to 1000 ml, 370 ml to 1000 ml, 400 ml to 1000 ml, 440 ml to 1000 ml, 470 ml to 1000 ml, 500 ml to 1000 ml, 550 ml to 1000 ml, or 1000 ml to 1500 ml of product. In embodiments, the plant-based composition, in particular the fermented plant-based composition, more particularly the plant-based food product, according to embodiments of the invention is provided in a sealed or sealable container or a bottle or a can containing about 1 oz to 12 oz, 2 oz to 12 oz, 3 oz to 12 oz, 4 oz to 12 oz, 5 oz to 12 oz, 6 oz to 12 oz, 12 oz, 15 oz, 17 oz, 20 oz, or 35 oz product by weight.

Preferably the plant-based composition, in particular the fermented plant-based composition, more particularly the plant-based food product, according to embodiments of the invention, may be stored, transported and/or distributed at a temperature of from 1° C. to 10° C. for at least about 30 days, at least about 60 days or at least about 90 days from packaging and remain suitable for consumption.

### Process for the Preparation of a Fermented Plant-Based Composition

The process for the preparation of a fermented plant-based composition according to the present embodiments is particularly suited for the preparation of a plant-based food product, in particular a fermented plant-based food product.

Thus, embodiments provide a process for the preparation of a fermented plant-based composition comprising the steps of:
a) providing a mixture comprising a plant base comprising at least 5% w/w protein and at least 1% w/w fat;
b) hydrolysis with at least one protease;
c) fermenting the mixture;
wherein the plant-based composition has a viscosity greater than 800 mPa·s.

Preferably, the plant base is as defined above.

Preferably, the protease is as defined above.

Preferably, the process as defined above may further comprise at least one step d) of heat treatment before of after hydrolysis.

Preferably, the process as defined above may further comprise at least one step e) of concentration before or after the fermentation step.

Preferably, the process as defined above may further comprise at least one step f) of homogenization, before or after step c) of fermentation.

Preferably, the process as defined above further comprise a step g) of inactivation of the at least one protease after the step b) of hydrolysis.

Preferably, the process according to the present embodiments may further comprise a step of adding at least one buffering agent.

Preferably, the process according to the present embodiments may further comprise a step of adding water.

Preferably, the process according to the present embodiments may further comprise a step of adding at least one intermediate preparation, in particular at least one flavoring agent, at least one sweetener, at least one fruit or at least one fruit preparation, at least one stabilizing agent, at least one coloring agents, at least one vitamin, oilseeds, nuts, cereals, and/or mixtures and combinations thereof.

Preferably, the process according to the present embodiment may further comprise a step of packaging, bottling, or filling components.

### Hydrolysis

Preferably, the step b) of hydrolysis comprises the mixing of the mixture comprising a plant base comprising at least 5% w/w protein and at least 1% w/w fat according to the present embodiments with at least one protease.

Preferably, the mixing can be carried out by any methods well known by the person skilled in the art. By way of example, the mixing may include one or more means of blending, mixing, combining, stirring, and/or agitating ingredients such as shear mixer, high shear rate mixer, food processor, stirrer, etc.

Preferably, the hydrolysis step b) is performed at a temperature comprised between 35° C. and 60° C., for example during 10 to 80 minutes for example at a pH comprised between 5 and 8. The optimal conditions of action of the at least one protease according to the present embodiments can be easily determined by the person skilled in the art.

In an embodiment, the step b) of hydrolysis is performed before the step c) of fermentation.

In another embodiment, the step b) of hydrolysis is performed during the step c) of fermentation. As intended herein, when the step b) of hydrolysis is performed during the step c) of fermentation this means that the hydrolysis and the fermentation steps are performed in the same time or at least overlap.

### Fermentation

Preferably, during the fermentation step c), the process mixture is allowed to ferment at a fermentation temperature to obtain a fermented mixture. The fermentation operations are well known by the person skilled in the art. Preferably the fermentation temperature is comprised between 27° C. and 60° C., more preferably between 35° C. to 45° C.

In an embodiment, the process as defined above comprises step a) of providing a mixture comprising a plant base comprising at least 5% w/w protein and at least 1% w/w fat; then step b) of hydrolysis with at least one protease; then a step c) fermenting the mixture.

In another particular embodiment, the process as defined above comprises step a) of providing a mixture comprising a plant base comprising at least 5% w/w protein and at least 1% w/w fat; then step c) fermenting the mixture; then step b) of hydrolysis with at least one protease.

In another particular embodiment, the process as defined above comprises step a) of providing a mixture comprising a plant base comprising at least 5% w/w protein and at least 1% w/w fat; then the step b) and the step c) are performed at the same time or at least overlap. In an embodiment, the mixture as defined in step a) is incubated with lactic acid bacteria and until 2 to 20 minutes, preferably 5 to 15 minutes, after the start of the fermentation, the protease is added to the mixture which is then allowed to ferment while the protease hydrolyses the peptide bonds. In another embodiment, the lactic acid bacteria and the at least in protease are introduced in the same time in the mixture.

### Heat Treatment

Heat treatments are known by the person skilled in the art as, for example, pasteurization or sterilization. Preferably, heat treatments are used to eliminate micro-organism contaminants such as, for example, bacteria. Heat treatments may be performed in conventional heat exchangers, such as tube or plate heat exchangers.

Preferably, the heat treatment step is performed before or after step b) of hydrolysis.

In on embodiment, the process as defined above may comprise at least one step of heat treatment preferably before the step c) of fermentation. The heat treatment may, for example, be performed at a temperature of from 80° C. to 150° C., for example, during 2 seconds to 10 minutes.

In another embodiment, the process as defined above may comprise at least one step of heat treatment before the step b) of hydrolysis with at least on protease, in particular when step b) of hydrolysis and step c) of fermentation are performed at the same time.

### Homogenization

Homogenization of the mixture according to the present embodiments may be performed by any method well-known by the person skilled in the art. The homogenization can be a single step homogenization or a double step homogenization.

### Concentration

The concentration according to the present embodiments may be performed according to any well-known concentration method.

Typically, the concentration step provides a plurality of fractions. By way of example of fractions which can be obtained, it is possible to cite a heavy phase and a light phase, such as a retentate phase and a permeate phase. Preferably, the higher density phase is rich in protein, starch, insoluble fibers and fat and the lower phase is rich in soluble proteins, peptides, carbohydrates, fiber, minerals, and other vegetable compounds.

Preferably, the concentration according to the present embodiments performed by ultrafiltration and/or centrifugation. Preferably, when centrifugation is used, the concentration step is performed after the fermentation step.

In one embodiment, the concentration step is performed after step c) of fermentation on the fermented mixture and produces a heavy phase or retentate comprising the concentrated fermented plant-based composition and a light phase, or “whey” or permeate. In some aspects, the concentration is carried out by membrane filtration. Membrane filtration technology uses one or more porous membrane filters (or selective barriers) to separate particles in a fluid. Particles may be separated on the basis of their size and shape with the application of a pressure differential to pass a mixture across the membrane. The part of the mixture retained by the membrane is termed the retentate or concentrate, the part of the mixture that passes across the membrane is termed the permeate. Specially designed membranes with different pore sizes may be used to target the separation of different particles. Additionally, pressure and temperature may be adjusted within or near the membrane filters to facilitate the separation of target particles.

In certain aspects, the composition is filtered through an ultrafiltration (UF) membrane having a maximum pore diameter between 0.01 and 0.001 microns. Alternatively, the membranes may be referred to by their molecular weight cut-off (MWCO) rather than pore size. Ultrafiltration membranes suitable for use in the methods of the present invention may have a MWCO in the range of 1,000-100,000 Daltons, more preferably 5,000-500,000 Daltons.

In particular aspects, filtering the composition with an ultrafiltration membrane includes filtering the composition to provide the plant-based composition of the invention which is the retentate phase comprising 5 to 20% w/w of plant protein and from 3 to 10% w/w fat.

Certain materials may be more effective in filtering various components from the composition. In certain aspects, the composition may be filtered through membrane filters including ceramic, organic, flat sheet, spiral wound poly ether sulfone.

In one embodiment, the concentration step is performed after step c) of fermentation. Thus, the concentration step is performed on the plant base and produce a retentate which forms the fermented plant-based food product of the invention.

In another embodiment, the concentration step is performed before step b) of hydrolysis. Thus, the concentration step is performed on the plant base and produce a retentate which form the mixture of step a). The retentate obtained by the concentration step can be used alone or can be mixed with a powder, or a milk as defined above.

Preferably, the concentration step is performed at a temperature comprised between 4° C. and 90° C.

In an embodiment, the concentration step is carried out such that the protein concentration in the mixture is multiplied by a factor of at least 1.5, such as at least 3.0 with reference to the protein concentration of mixture. The factor is preferably at most 5, for example from 1.5 to 5.0 or from 2.0 to 4.5 or from 2.5 to 4.

### Inactivation

The inactivation of the at least one protease can be performed by any method well-known by the person skilled in the art. By way of example of inactivation method according to the present embodiments, it is possible to cite heat treatment and/or pH modification and/or substrate depletion. Preferably, the at least one protease is inactivated after the hydrolysis step by at least two of the following parameters heat treatment, pH modification and substrate depletion.

Preferably, when the hydrolysis step b) is performed before fermentation, the at least one protease is inactivated by heat treatment before the fermentation step c). Preferably, the deactivation can take place at a temperature comprised between 85 and 150° C., for example between 9° and 150° C., 90 and 100° C., 95 and 120° C., 100 and 150° C., for example during 3 s to 5 min for example during 4 s to 60 s, or during 4 s to 10 s.

Preferably, when the hydrolysis step b) is performed during fermentation, the at least one protease is inactivated by the modification of the pH during the fermentation step. Preferably, the inactivation of the protease occurs at a pH comprised in the range of 3 to 8, for example 3 to 5 or 3.5 to 4.5 is used.

### Further Processing

In embodiments, the plant-based composition, in particular the fermented plant-based composition or the concentrated fermented plant-based composition, is recovered preferably after the step g) of inactivation of the protease.

The plant-based composition, in particular the fermented plant-based composition or the concentrated fermented plant-based composition, according to the present embodiments may optionally be cooled at a temperature comprised between 0° C. and 25° C., by way of example between 0° C. and 20° C., between 4° C. and 15° C., or between 5° C. and 10° C.

The process according to the present embodiments may optionally comprise a step of packaging, bottling, or filling the plant-based composition, in particular the fermented plant-based composition or the concentrated fermented plant-based composition, in one or more bottles, jars, cans, cartons, and/or any other appropriate container or in a form suitable for commercial sale or use.

The plant-based composition, in particular the fermented plant-based composition or the concentrated fermented plant-based composition, according to the present embodiments may also be processed for example to a further food form, such as for example smoothed.

The plant-based composition, in particular the fermented plant-based composition or the concentrated fermented plant-based composition, according to the present embodiments may also optionally be stored, by way of example in a tank.

The plant-based composition, in particular the fermented plant-based composition or the concentrated fermented plant-based composition, according to the present embodiments may also optionally be used as an ingredient involved in preparing a food.

In one embodiment, the plant-based composition according to the present embodiments are substantially free of and do not require the use of an emulsifying agent, such as mono- and diglycerides, polysorbates, carrageenan, guar gum and canola oil. Suitable lecithins, include but are not limited to, soy lecithin, sunflower lecithin, or rapeseed lecithin. In another embodiment, the compositions described herein are free of a lecithin. Should the composition comprise a minor amount of lecithin, this amount is less than 0.1% by weight, for example, less than 0.05%, for example, less than 0.01%, for example, less than 0.005%, for example, less than 0.001%.

The invention will be further described by the following non-limiting Figures and Examples.

### FIGURES

FIG. 1 is a diagram representing the characteristics i) texture smooth and creamy, ii) beany notes, iii) lumpy, and iv) acidity of plant-based alternative yogurts having 7% w/w protein and about 3% fat obtained according to the invention (Example A and Example B) and a plant-based alternative yogurt obtained without protease (Comparative example 1).

FIG. 2 is a diagram representing the characteristics i) texture smooth and creamy, ii) beany notes, iii) lumpy, and iv) acidity of plant-based alternative yogurts having 10% w/w protein and about 5% w/w fat obtained according to the invention (Example C and Example D) and a plant-based alternative yogurt obtained without protease (Comparative example 2).

FIG. 3 provides the particle size distribution of compositions prepared according to Example 3.

### EXAMPLES

### Example 1

The inventors have compared the texture and organoleptic properties of plant-based compositions, in particular plant-based alternative yogurts according the present invention prepared using the methods described below and a plant-based composition without protease.

### A. Material and Method

### 1. Preparation of Plant-Based Alternative Yogurt Comprising 7% w/w Protein

### 1.1. Example A

A plant base is prepared by mixing 3 to 4% of spray-dried soymilk powder with 95 to 96% of soy milk to obtain a mixture comprising about 7% w/w protein and about 3% w/w fat. The mixture is stirred for 30 minutes at a temperature of about 40° C.

Then 0.05-0.1% w/w of a protease ingredient which is an exopeptidase from the fungal source Aspergillus oryzae is added to the mixture. The mixture is then incubated at a temperature between 45 and 55° C. at a pH 7-7.5 for 1 hour and homogenized at 210 bars. The protease is then inactivated by heat treatment at a temperature of 95° C. for 6 minutes.

The mixture is then fermented with lactic acid bacteria at about 43° C. for 5 to 7 hours to provide an unflavoured yogurt type product.

The plant-based alternative yogurt is recovered and allowed to cool at a temperature of 4-10° C.

### 1.2. Example B

A plant base is prepared by mixing 3 to 4% of spray-dried soymilk powder with 95 to 96% of soy milk to obtain a mixture comprising about 7% w/w protein and about 3% w/w fat. The mixture is stirred for 30 minutes at a temperature of about 40° C. and homogenized at 201 bars. The mixture is then heat treated at 95° C. for 6 minutes.

Then, the mixture is fermented with lactic acid bacteria and a protease which is a microbial endoprotease is added during fermentation of the mixture about 10 minutes after the beginning of the fermentation. The protease is then inactivated with a pH of about 4-4.6.

The plant-based composition is then recovered and allowed to cool at a temperature of 4-10° C.

### 1.3. Comparative Example 1

A plant-based composition comprising 7% w/w protein and about 3% fat has been prepared using the same protocol as disclosed in point 1.1. but without the step of incubating the mixture with a protease.

### 2. Preparation of Plant-Based Alternative Yogurt Comprising 10% w/w Protein

### 2.1. Example C

A plant base is prepared by mixing 10 to 11% of spray-dried soymilk powder with 88 to 89% of soy milk to obtain a mixture comprising about 10% w/w protein and about 5% w/w fat. The mixture is stirred for 30 minutes at a temperature of about 40° C.

Then 0.05-0.1% w/w of a protease ingredient which is an exopeptidase from the fungal source Aspergillus oryzae is added to the mixture. The mixture is then incubated at a temperature between 45 and 55° C. at a pH between 5 and 7 for 1 hour and homogenized at 210 bars. The protease is then inactivated by heat treatment at a temperature of 80° C. for 4 to 10 seconds.

The mixture is then fermented with lactic acid bacteria at about 43° C. for 5 to 7 hours to provide an unflavoured yogurt type product.

The plant-based alternative yogurt is then recovered and allowed to cool at a temperature of 4-10° C.

### 2.2. Example D

A plant base is prepared by mixing 10 to 11% of spray-dried soymilk powder with 88 to 89% of soy milk to obtain a mixture comprising about 10% w/w protein and about 5% w/w fat. The mixture is stirred for 30 minutes at a temperature of about 40° C. and homogenized at 201 bars. The mixture is then heat treated at 95° C. for 6 minutes.

The mixture is then fermented with lactic acid bacteria and a protease which is a microbial endoprotease is added during fermentation of the mixture about 10 minutes after the beginning of the fermentation. The protease is then inactivated with a pH of about 4-4.6.

The plant-based composition is then recovered and allowed to cool at a temperature of 4-10° C.

### 2.3. Comparative Example 2

A plant-based composition comprising about 10% w/w protein and about 5% w/w fat has been prepared using the same protocol as disclosed in point 2.1. but without the step of incubating the mixture with a protease.

### B. Analysis

### 1. Sensory Test

A sensory test has been performed by a panel of trained testers to compare the plant-based compositions according to the present invention (Example A to Example D) and the plant-based composition obtained without the use of protease (Comparative Examples 1 and 2).

The testers have attributed a note from 0 to 5 for each of the following feature: beany notes, lumpy texture, smoothness and creaminess. The note 0 is attributed when the feature is not felt. The note 5 is attributed when the feature is strongly felt.

### 2. Viscosity

The viscosity of the plant-based compositions according to the invention (Example A to Example D) has been determined using the rheometer Anton Paar MCR 302 (64 s⁻¹; 10° C., 10-90 sec). The viscosity of the plant-based composition obtained without the use of protease (Comparative Examples 1 and 2) has not been measured because the compositions was too thick and not processable.

### C. Results

### 1. Sensory Test

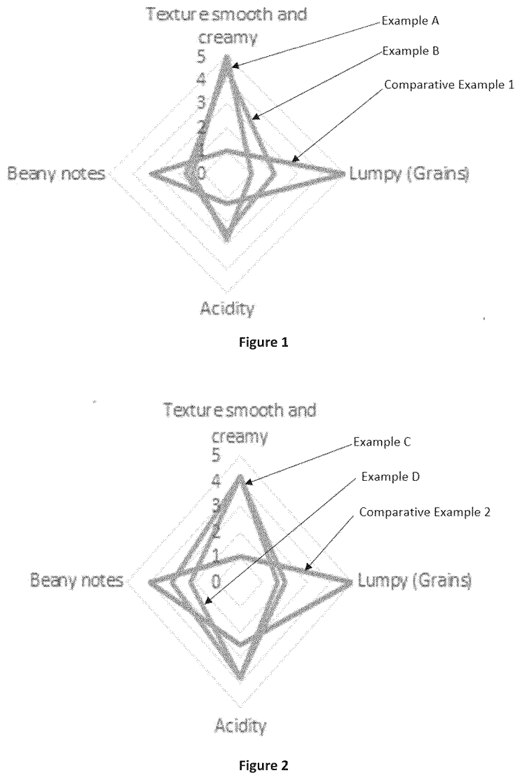

Results of the sensory test are presented in FIG. 1 and FIG. 2. The panel of testers has found that the plant-based composition according to the invention (Example A to Example D) have a smooth and creamy texture with a dairy mouthfeel and no taste defects at all protein levels (7% w/w and 10% w/w). This result equally applied for both processes used i.e., with the hydrolysis before the fermentation step or during the fermentation step.

In particular, the panel of testers has found that the plant-based composition according to the invention (Example A to Example D) have a reduced bean taste compare to the plant-based composition obtained without the use of protease (Comparative Examples 1 and 2). Furthermore, the plant-based compositions according to the invention have a spoonable texture.

In contrast, the panel of tester has found that the plant-based composition obtained without the use of protease has unpleasant bean notes and a strong uncomfortable lumpy texture. In addition, the plant-based composition obtained without the use of protease are too thick and are not spoonable.

### 2. Viscosity

The plant-based compositions according to the invention (Example A to Example D) have all a spoonable texture and a smooth and very creamy texture. The viscosity measured for the plant-based compositions according to the invention is comprised between 1113 and 1309 mPa·s for Example A and Example B and between 820 and 1250 for Example C and Example D.

The viscosity of the plant-based composition obtained without the use of protease cannot be determined because this composition was not processable.

### Example 2

A concentrated fermented plant-based composition according the present invention having 10% w/w protein has been prepared using the methods described below.

### A. Materials and Methods

### 1. Example E

A concentration step using ultrafiltration method is performed on a soy juice comprising about 5.5% w/w protein, 2.5% w/w fat at ambient temperature to obtained a retentate of soy juice. Then about 97.5% w/w of the retentate of soy juice is mixed with about 2.5% w/w sucrose. The mixture is heat treated by direct steam injection at a temperature comprised between 85° C. and 122° C. for 8 seconds and the mixture is homogenized at 50 bars.

The mixture is then fermented with lactic acid bacteria and a protease which is a microbial endoprotease is added to the mixture during fermentation. The fermentation step is performed during 6-8 hours at a temperature of about 43° C. The protease is then inactivated with a pH of about 4.6.

Then a smoothing step is performed, and the concentrated fermented plant-based composition having 10% w/w protein is recovered and allowed to cool at a temperature of 4-10° C.

### 2. Example F

A soy juice comprising about 5.5% w/w protein and about 2.5% w/w fat is heat treated by indirect steam injection at a temperature comprised between 85° C. and 118° C. for 15 seconds and homogenized at 0 bar at a temperature of 43° C.

The mixture is then fermented with lactic acid bacteria and a protease which is a microbial endoprotease is added to the mixture during fermentation. The fermentation step is performed during 6-8 hours at a temperature of about 43° C. The protease is then inactivated with a pH of about 4.6.

Then, a step of concentration by ultrafiltration is performed on the fermented mixture at ambient temperature. The retentate comprising 10% w/w protein and 5.3% w/w fat is recovered is subjected to a smoothing step.

Then, the concentrated fermented plant-based composition having 10% w/w protein is recovered and allowed to cool at a temperature of 4-10° C.

### B. Analysis

### 1. Sensory Test

A sensory test has been performed by a panel of trained testers. The testers have evaluated the beany notes, lumpy texture, smoothness and creaminess of the compositions.

### 2. Results

The panel of testers has found that the plant-based composition according to the invention (Example E and F) have a smooth and creamy texture with a dairy mouthfeel and no taste defects. This result equally applied for both processes used i.e., with the concentration step performed before or after the fermentation step.

Furthermore, the plant-based compositions according to the invention have a spoonable texture.

### Example 3

A soy base was prepared by processing whole soy beans with water to provide a “juice type” base comprising 5.4% protein and 2.5% fat. The juice was concentrated by ultrafiltration to provide a retentate base comprising 10.3% protein and 5.3% fat, sugar was added to provide a liquid composition suitable for the preparation of fermented plant-based yogurt alternative.

The liquid composition was then hydrolysed enzymatically and heat treated as above and optionally homogenized. Three different formulations were tested:
A: enzymatic hydrolysis 30 mins.
B: enzymatic hydrolysis 60 mins.
C: enzymatic hydrolysis 60 mins+homogenization 300 bars at 4° C.

A reduction in viscosity and sedimentation was observed in all 3 samples.

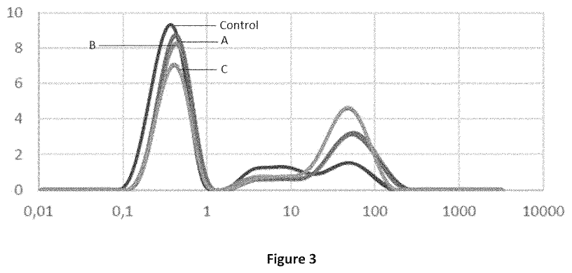

Granulometry analysis was carried out on all 3 samples to better understand the mechanisms behind the enzymation and homogenization processes. A non-enzymatically treated plant-based beverage was used as control. The Inventors noted an increase in particles in the 10-100 microns range with enzymation (and also in the homogenized sample), see FIG. 3, corresponding to the fat range and without wishing to be bound theory hypothesized that these correspond to an agglomeration of peptides with the fat molecules. Such peptides would be less likely to be passed into the permeate stream, improving the preparation of high protein products.

1. A plant-based composition comprising a mixture comprising:
i) a plant base comprising at least 5% w/w protein and at least 1% w/w fat; and
ii) at least one protease,
wherein the plant-based composition has a viscosity greater than 800 mPa·s.

2. The plant-based composition according to claim 1, wherein the plant-based composition has a viscosity between 800 and 3500 mPa·s.

3. The plant-based composition according to claim 1, further comprising at least one strain of lactic acid bacteria.

4. The plant-based composition according to claim 1, comprising from 5 to 20% w/w of plant protein and from 1 to 10% w/w fat.

5. The plant-based composition according to claim 1, further comprising at least one buffering agent.

6. The plant-based composition according to claim 1, wherein the plant protein is selected from the group consisting of soy protein, pulse protein, cereal protein, nut protein and mixtures thereof.

7. A process for the preparation of a plant-based composition comprising the step of:
a) proving a mixture comprising a plant base comprising at least 5% w/w protein and at least 1% w/w fat;
b) hydrolysis with least one protease; and
c) fermenting the mixture,
wherein the plant-based composition has a viscosity greater than 800 mPa·s.

8. The process according to claim 7, further comprising at least one step d) of heat treating before or after step b) of hydrolysis.

9. The process according to claim 7, further comprising at least one step e) of concentrating before or after fermentation.

10. The process according to claim 9, wherein the concentrating step e) is carried out by ultrafiltration or centrifugation.

11. The process according to claim 7, further comprising at least one step g) of inactivating the at least one protease.

12. The process according to claim 11, wherein the protease is inactivated by heat treatment or pH modification.

13. The process according to claim 7, wherein the hydrolysis step b) is performed before or during the fermentation step c).

14. A plant-based composition obtained by the process of claim 7.

15. A plant-based dairy alternative comprising the plant-based composition of claim 1.
